(19) United States
(12) Patent Application Publication
(10) Pub. No.: US 2026/0151103 A1
(43) Pub. Date: Jun. 4, 2026

(54) INTRAVASCULAR IMAGING CATHETER

(71) Applicant: Boston Scientific Scimed, Inc., Maple Grove, MN (US)

(72) Inventors: Jeffrey Steven Fuller, Brooklyn Park, MN (US); Brice Lee Shireman, Maple Grove, MN (US)

(73) Assignee: Boston Scientific Scimed, Inc., Maple Grove, MN (US)

(21) Appl. No.: 19/403,560

(22) Filed: Nov. 28, 2025

Related U.S. Application Data
(60) Provisional application No. 63/726,414, filed on Nov. 29, 2024.

(51) Int. Cl. A61B 8/12; A61B 8/00; A61B 8/08

(52) U.S. Cl. CPC A61B 8/12; A61B 8/0891; A61B 8/445

(57) ABSTRACT

Intravascular imaging devices as well as methods for making and using intravascular imaging devices are disclosed. An intravascular imaging device may include a catheter shaft including a hypotube region and an imaging window region. The hypotube region may include a slotted section having a plurality of slots formed therein. The slotted section may include a plurality of discrete zones including a first zone, a second zone disposed proximal of the first zone, a third zone disposed proximal of the second zone, a fourth zone disposed proximal of the third zone. An imaging core may be disposed within the catheter shaft.

### CROSS-REFERENCE TO RELATED APPLICATIONS

This application claims the benefit of priority under 35 U.S.C. § 119 of U.S. Provisional Application No. 63/726,414, filed Nov. 29, 2024, the entire disclosure of which is hereby incorporated by reference.

### TECHNICAL FIELD

The present disclosure pertains to medical devices, and methods for manufacturing medical devices. More particularly, the present disclosure pertains to intravascular imaging catheters.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An intravascular imaging device is disclosed. The intravascular imaging device comprises: a catheter shaft including a hypotube region and an imaging window region; wherein the hypotube region includes a slotted section having a plurality of slots formed therein; wherein the slotted section includes a plurality of discrete zones including a first zone, a second zone disposed proximal of the first zone, a third zone disposed proximal of the second zone, a fourth zone disposed proximal of the third zone; wherein the slots along the first zone have a constant first zone pitch; wherein the slots along the second zone have a second zone pitch that varies along a second zone length; wherein the slots along the third zone have a constant third zone pitch; wherein the slots along the fourth zone have a fourth zone pitch that varies along a fourth zone length; and an imaging core disposed within the catheter shaft.

Alternatively or additionally to any of the embodiments above, further comprising a distal end zone disposed distally of the first zone.

Alternatively or additionally to any of the embodiments above, the distal end zone is free of slots.

Alternatively or additionally to any of the embodiments above, the slots along the first zone have a constant first cut angle.

Alternatively or additionally to any of the embodiments above, the constant first cut angle is about 150-160 degrees.

Alternatively or additionally to any of the embodiments above, the slots along the second zone have a constant second cut angle.

Alternatively or additionally to any of the embodiments above, the constant second cut angle is about 150-160 degrees.

Alternatively or additionally to any of the embodiments above, the slots along the third zone have a cut angle that varies along a third zone length thereof.

Alternatively or additionally to any of the embodiments above, the cut angle of the slots adjacent to a distal end of the third zone is about 140-150 degrees and wherein the cut angle of the slots adjacent to a proximal end of the third zone is about 70-75 degrees.

Alternatively or additionally to any of the embodiments above, the slots of the fourth zone have a cut angle that varies along a fourth zone length thereof.

Alternatively or additionally to any of the embodiments above, the cut angle of the slots adjacent to a distal end of the fourth zone is about 70-75 degrees and wherein the cut angle of the slots adjacent to a proximal end of the fourth zone is about 40-50 degrees.

Alternatively or additionally to any of the embodiments above, the catheter shaft includes a distal end region having a guidewire lumen formed therein.

An intravascular imaging device is disclosed. The intravascular imaging device comprises: a catheter shaft including a hypotube region; wherein the hypotube region includes a slotted section having a plurality of slots formed therein; wherein the slotted section includes a plurality of discrete zones including a first zone, a second zone disposed proximal of the first zone, a third zone disposed proximal of the second zone, a fourth zone disposed proximal of the third zone; wherein the slots along the first zone have a constant first zone pitch; wherein the slots along the second zone have a second zone pitch that varies along a second zone length; wherein the slots along the third zone have a constant third zone pitch; and wherein the slots along the fourth zone have a fourth zone pitch that varies along a fourth zone length.

Alternatively or additionally to any of the embodiments above, further comprising a distal end zone disposed distally of the first zone.

Alternatively or additionally to any of the embodiments above, the distal end zone is free of slots.

Alternatively or additionally to any of the embodiments above, the slots along the first zone have a constant first cut angle.

Alternatively or additionally to any of the embodiments above, the constant first cut angle is about 150-160 degrees.

Alternatively or additionally to any of the embodiments above, the slots along the second zone have a constant second cut angle.

Alternatively or additionally to any of the embodiments above, the constant second cut angle is about 150-160 degrees.

Alternatively or additionally to any of the embodiments above, the slots along the third zone have a cut angle that varies along a third zone length thereof.

Alternatively or additionally to any of the embodiments above, the cut angle of the slots adjacent to a distal end of the third zone is about 140-150 degrees and wherein the cut angle of the slots adjacent to a proximal end of the third zone is about 70-75 degrees.

Alternatively or additionally to any of the embodiments above, the slots of the fourth zone have a cut angle that varies along a fourth zone length thereof.

Alternatively or additionally to any of the embodiments above, the cut angle of the slots adjacent to a distal end of the fourth zone is about 70-75 degrees and wherein the cut angle of the slots adjacent to a proximal end of the fourth zone is about 40-50 degrees.

Alternatively or additionally to any of the embodiments above, the catheter shaft includes an imaging window region.

Alternatively or additionally to any of the embodiments above, the catheter shaft includes a distal end region having a guidewire lumen formed therein.

Alternatively or additionally to any of the embodiments above, further comprising an imaging core disposed within the catheter shaft.

An intravascular imaging device is disclosed. The intravascular imaging device comprises: a catheter shaft including a hypotube region, an imaging window region, and a distal end region having a guidewire lumen formed therein; wherein the hypotube region includes a slotted section having a plurality of slots formed therein; wherein the slotted section includes a plurality of discrete zones including a first zone, a second zone disposed proximal of the first zone, a third zone disposed proximal of the second zone, a fourth zone disposed proximal of the third zone; wherein the slots along the first zone have a constant first cut angle; wherein the slots along the second zone have a constant second cut angle; wherein the slots along the third zone have a third cut angle that varies along a third zone length thereof; wherein the slots along the fourth zone have a fourth cut angle that varies along a fourth zone length thereof; and an imaging core disposed within the catheter shaft.

Alternatively or additionally to any of the embodiments above, the slots along the first zone have a constant first zone pitch.

Alternatively or additionally to any of the embodiments above, the slots along the second zone have a second zone pitch that varies along a second zone length.

Alternatively or additionally to any of the embodiments above, the slots along the third zone have a constant third zone pitch.

Alternatively or additionally to any of the embodiments above, the slots along the fourth zone have a fourth zone pitch that varies along a fourth zone length.

Alternatively or additionally to any of the embodiments above, the third cut angle of the slots adjacent to a distal end of the third zone is about 140-150 degrees and wherein the third cut angle of the slots adjacent to a proximal end of the third zone is about 70-75 degrees.

Alternatively or additionally to any of the embodiments above, the fourth cut angle of the slots adjacent to a distal end of the fourth zone is about 70-75 degrees and wherein the fourth cut angle of the slots adjacent to a proximal end of the fourth zone is about 40-50 degrees.

Alternatively or additionally to any of the embodiments above, further comprising a distal end zone disposed distally of the first zone, the distal end zone being free of slots.

A method for imaging a blood vessel is disclosed. The method comprises: disposing an intravascular imaging device within a blood vessel, the intravascular imaging device comprising: a catheter shaft including a hypotube region, an imaging window region, and a distal end region having a guidewire lumen formed therein, wherein the hypotube region includes a slotted section having a plurality of slots formed therein, wherein the slotted section includes a plurality of discrete zones including a first zone, a second zone disposed proximal of the first zone, a third zone disposed proximal of the second zone, a fourth zone disposed proximal of the third zone, wherein the slots along the first zone have a constant first zone pitch, wherein the slots along the second zone have a second zone pitch that varies along a second zone length, wherein the slots along the third zone have a constant third zone pitch, wherein the slots along the fourth zone have a fourth zone pitch that varies along a fourth zone length, and an imaging core disposed within the catheter shaft; and translating the imaging core relative to the catheter shaft.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of a portion of an example medical device.

FIG. 2 is a side view of an example medical device.

FIG. 3 is a partial cross-sectional side view of a portion of an example medical device.

FIG. 4 schematically illustrates characteristics/arrangements of slots.

FIG. 5 is a partial cross-sectional side view of a portion of an example medical device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

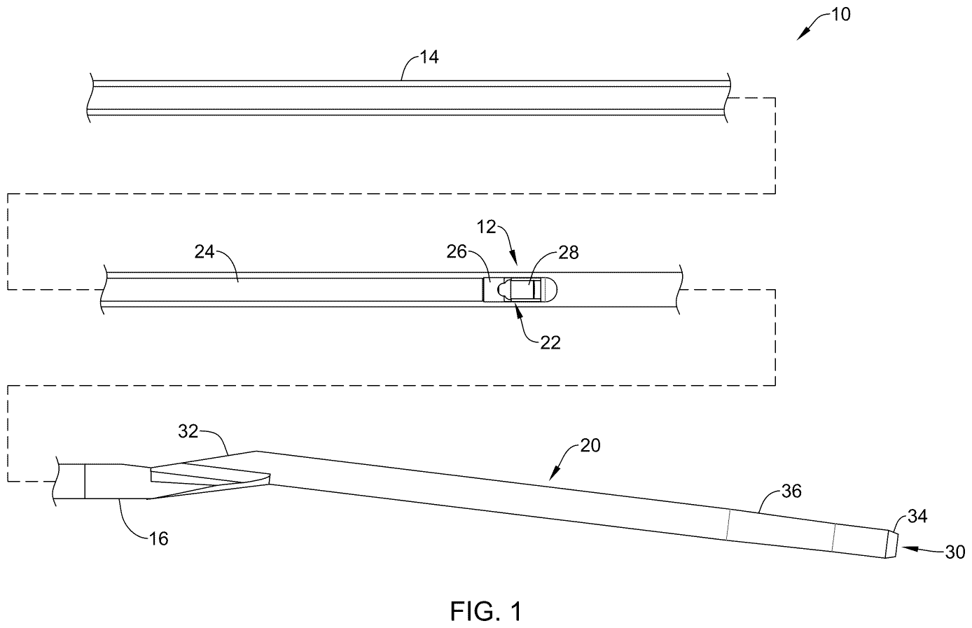

FIG. 1 is a side view of a portion of an example medical device 10. In at least some instances, the medical device 10 takes the form of an imaging medical device. For example, the medical device 10 may be an intravascular ultrasound (IVUS) device that may be used to image a blood vessel. In some of these and in other instances, the medical device may be an optical coherence tomography (OCT) imaging device, a near-infrared spectroscopy (NIRS) imaging device, a near-infrared fluorescence (NIRF) imaging device, a photoacoustic imaging device, a fluorescence-lifetime imaging device, combinations thereof, and/or the like. The structure/form of the medical device 10 can vary. In some instances, the medical device 10 may include an elongate shaft 12 having a proximal end region 14 and a distal end region 16. A tip member 20 may be coupled to or otherwise disposed adjacent to the distal end region 16. The tip member 20 may include a guidewire lumen 30 having a guidewire exit port 32, an atraumatic distal end 34, one or more radiopaque markers 36, and/or other features. In some embodiments, the tip member 20 may extend at a non-parallel angle to the proximal end region 14 of the elongate shaft 12.

An imaging assembly 22 (e.g., which may sometime be referred to as an imaging core) may be disposed within a lumen of the elongate shaft 12. In general, the imaging assembly 22 may be used to capture/generate images of a blood vessel. In some instances, the medical device may include devices and/or features similar to those disclosed in U.S. Patent Application Pub. No. US 2012/0059241 and U.S. Patent Application Pub. No. US 2017/0164925, the entire disclosures of which are herein incorporated by reference. In at least some instances, the medical device 10 may resemble and/or include features that resemble the OPTICROSS™ Imaging Catheter, commercially available from BOSTON SCIENTIFIC, Marlborough, MA.

The imaging assembly 22 may include a drive shaft or cable 24, a housing 26, and an imaging member or transducer 28 coupled to the drive shaft 24 and/or housing 26. In at least some instances, the transducer 28 includes an ultrasound transducer. Other transducers are also contemplated. The transducer 28 may be rotatable and/or axially translatable relative to the elongate shaft 12. For example, the drive shaft 24 may be rotated and/or translated in order to rotate and/or translate the transducer 28 (and the housing 26).

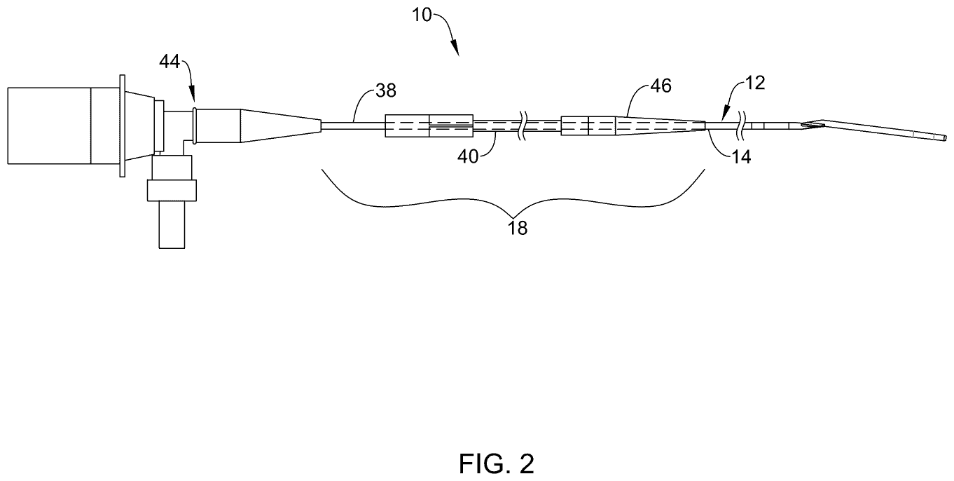

The proximal end region 14 of the elongate shaft 12 may be coupled to a telescoping assembly 18 as shown in FIG. 2. In general, the telescoping assembly 18 may be configured to allow the medical device operator to move the drive shaft 24 including the imaging assembly 22 proximally and distally within the catheter (e.g., relative to the elongate shaft 12), without having to move the entire catheter within the patient. This allows the catheter operator to easily change the location of the imaging assembly or other medical device within the patient. For example, the telescoping assembly 18 may be actuated to change the location of the imaging assembly 22 within the elongate shaft 12.

The proximal end region 14 of the elongate shaft 12 may be coupled to the telescoping assembly 18. For example, the proximal end region 14 of the elongate shaft 12 may be coupled to a distal hub 46 of the telescoping assembly 18. A proximal hub 44 may be coupled to the telescoping assembly 18 (e.g., at the proximal end of the telescoping assembly 18). The drive shaft 24 (see FIG. 1) may extend through the telescoping assembly 18 and be coupled to and/or otherwise secured to the proximal hub 44.

The telescoping assembly 18 may include a first sheath 38 and a second sheath 40. In some instances, the first sheath 38 may be understood to be an inner telescoping tube 38 and the second sheath 40 may be understood to be an outer telescoping tube 40. Generally, the outer telescoping tube 40 may be disposed over the inner telescoping tube 38. The inner telescoping tube 38 may be coupled to or otherwise secured to the proximal hub 44. The outer telescoping tube 40 may be coupled or otherwise secured to the distal hub 46. The inner telescoping tube 38 may be axially and/or rotatably moveable relative to the outer telescoping tube 40. Because the drive shaft 24 may be secured to the proximal hub 44 and/or the inner telescoping tube 38 and because the elongate shaft 12 may be secured to the distal hub 46, movement of the proximal hub 44 relative to the distal hub 46 results in movement of the inner telescoping tube 38 and the drive shaft 24 relative to the distal hub 46 and/or the elongate shaft 12.

In use, the elongate shaft 12 may be disposed within a target region (e.g., a blood vessel) and the imaging assembly 22 may be translated within the elongate shaft 12 in order to image the blood vessel. It can be appreciated that navigating the elongate shaft 12 through the vasculature toward the target region may include navigating the elongate shaft 12 through a number of tortuous bends and turns. As such, it may be desirable for the elongate shaft 12 to be sufficiently flexible in order to navigate such anatomy. Furthermore, it may be desirable for the elongate shaft 12 to be sufficiently pushable (e.g., in a manner that resists buckling) and be capable of transmitting torque along the length of the elongate shaft 12. Disclosed herein are medical devices (e.g., such as the medical device 10) where the elongate shaft 12 is designed to have a desirable level of flexibility, pushability, torquability, and/or other characteristics.

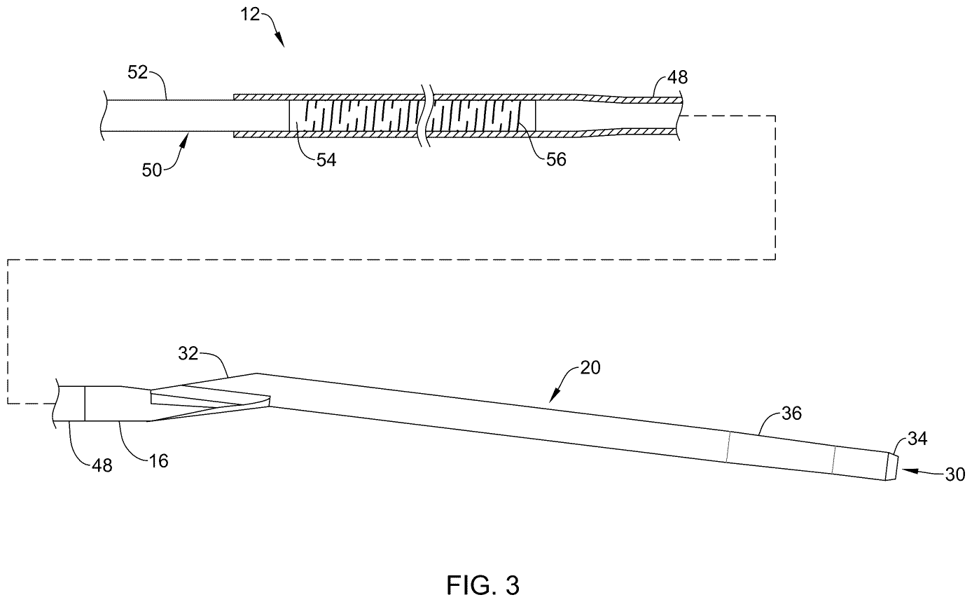

FIG. 3 illustrate a portion of the elongate shaft 12. As shown and described, the elongate shaft 12 may be an assembly of different parts/regions (e.g., the elongate shaft 12 may be understood to be a catheter shaft or catheter shaft assembly). For example, the elongate shaft 12 may include an imaging window region 48. As the name suggests, the imaging window region 48 is a region of the elongate shaft 12 through which the imaging assembly 22 (e.g., the transducer 28) can image through. While the imaging assembly 22 is not shown in FIG. 3, it can be appreciated that the imaging assembly 22 may be disposed within the elongate shaft 12 in the manner depicted in FIG. 1, for example. The imaging window region 48 may have a distal end that is coupled to and/or otherwise disposed adjacent to the tip member 20. In some instances, the imaging window region 48 extends the full length of the elongate shaft 12 (e.g., the full length proximally from the tip member 20). In other instances, the imaging window region 48 may extend along a portion of the elongate shaft 12. For example, the imaging window region 48 may have a length of about 5-50 cm (1.97-19.7 inches), or about 10-30 cm (3.94-11.8 inches), or about 15-25 cm (5.91-9.84 inches), or about 20-22 cm (7.87-8.66 inches). The imaging window region 48 may be formed from a suitable material such as nylon, nylon-12, polyether block amide, combinations thereof, and/or other suitable materials including those materials disclosed herein.

The elongate shaft 12 may also include a hypotube region 50. The hypotube region 50 may extend proximally from the imaging window region 48 to the distal hub 46. The hypotube region 50 may include a first portion 52 a second portion 54. The first portion 52 may be free of slots. The second portion 54 may have a plurality of slots 56 formed therein. The slots 56 are geometrically arranged apertures that are manufactured into the second portion 54 and may help to provide a desirable level of flexibility (and/or pushability and/or torqueability) of the elongate shaft 12. Various arrangements and configurations are contemplated for slots 56. For example, in some embodiments, at least some, if not all of the slots 56 are disposed at the same or a similar angle with respect to the longitudinal axis of the hypotube region 50. In some instances, the slots 56 can be disposed at an angle that is perpendicular, or substantially perpendicular, and/or can be characterized as being disposed in a plane that is normal to the longitudinal axis of the hypotube region 50. However, in other instances, the slots 56 can be disposed at an angle that is not perpendicular, and/or can be characterized as being disposed in a plane that is not normal to the longitudinal axis of the hypotube region 50. This may include angled slots 56, slots 56 arranged in a spiral or helical pattern/arrangement, and/or the like. Additionally, a group of one or more slots 56 may be disposed at different angles relative to another group of one or more slots 56. The distribution and/or configuration of the slots 56 can also include, to the extent applicable, any of those disclosed in U.S. Pat. Publication No. US 2004/0181174, the entire disclosure of which is herein incorporated by reference.

The slots 56 can be formed by methods such as micro-machining, saw-cutting (e.g., using a diamond grit embedded semiconductor dicing blade), electrical discharge machining, grinding, milling, casting, molding, chemically etching or treating, or other known methods, and the like. In at least some embodiments, the slots 56 may be formed in tubular member using a laser cutting process. The laser cutting process may include a suitable laser and/or laser cutting apparatus. In some instances, it may be desirable for the slots 56 to be arranged in the hypotube region 50 in a manner that helps to provide desirable flexibility and other characteristics. This may include arrangements of the slots 56 that are configured to provide flexibility characteristics that allow the device 10 to navigate the anatomy. For example, as indicated herein, the first portion 52 of the hypotube region 50 may be free of slots. The second portion 54 of the hypotube region 50 may include slots 56 along one or more sections/regions of its length. In some instances, the second portion 54 (e.g., slotted section) of the hypotube region 50 may include a plurality of discrete and/or distinct zones. For example, one or more of the different zones may include or be free of slots 56. The different zones may differ in length. The different zones may differ in the angle that the slots 56 are cut into the second portion 54. The different zones may differ in the pitch or spacing of the slots 56. These differences, which may be combined in different hypotube regions 50, may help to provide desirable characteristics.

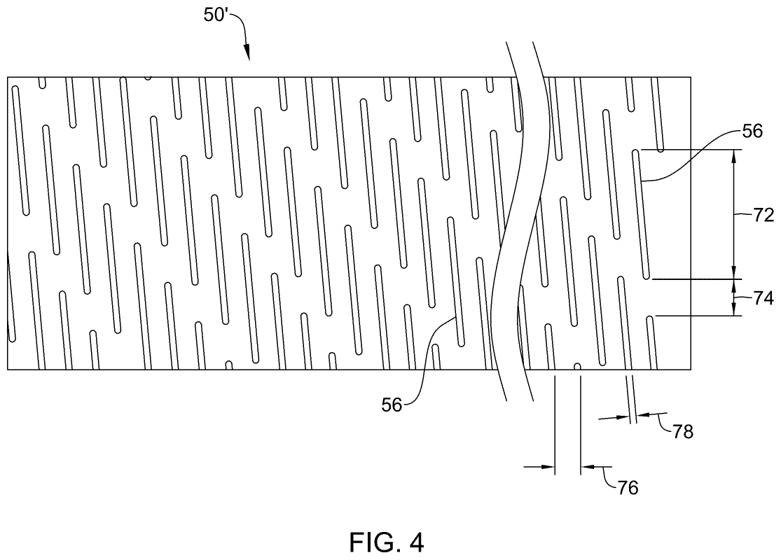

Some example characteristics/arrangements of the slots 56, and/or slots in general, are schematically depicted in FIG. 4. Here an example hypotube region 50′ is shown with slots 56 formed therein. The slots 56 may be arranged in a pattern that could be described as an interrupted spiral pattern. The slots 56 may have or define a cut angle 72 corresponding to the number of degrees the cut forming a slot 56 extends about the circumference of the hypotube region 50′. The slots 56 may also have or define an uncut angle 74 corresponding to the number of degrees an uncut region between slots 56 (e.g., spirally-patterned slots 56 that would connect if not interrupted by the uncut region) extend about the circumference of the hypotube region 50′. The slots 56 may also have a pitch 76, corresponding to the spacing between axially-adjacent slots 56. Finally, the slots 56 may have or define a kerf width 78, corresponding to the width of the slots 56.

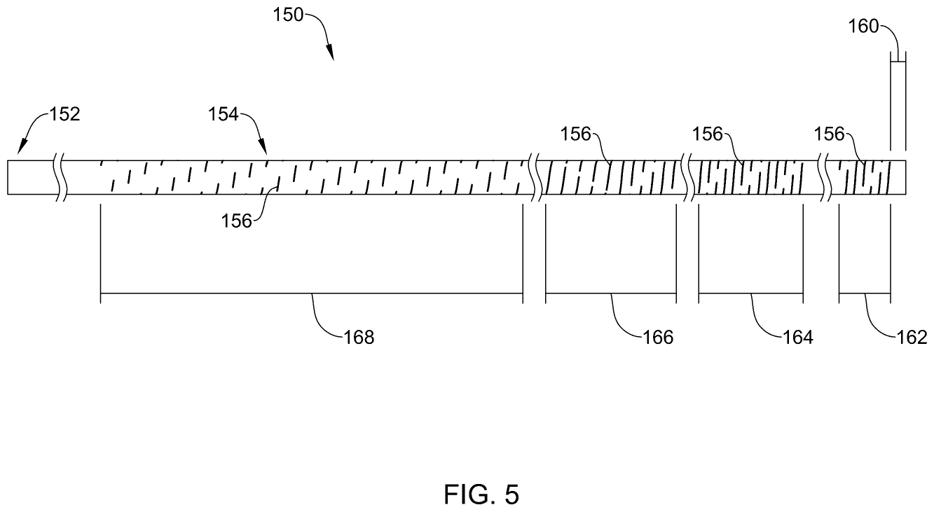

FIG. 5 illustrates a portion of an example hypotube region 150 that may be similar in form and function to other hypotube regions disclosed herein and/or may be used with any of the devices disclosed herein. For example, the hypotube region 150 may be used in place of the hypotube region 50 in the medical device 10. Thus, medical devices are contemplated that are similar in form and function to the medical device 10, but that utilize the hypotube region 150 disclosed herein. The hypotube region 150 may include a first portion 152 and a second portion 154. The first portion 152 may be free of slots. In at least some instances, the first portion 152 may have a length of about 1000-1500 mm (about 39-59 inches), or about 1200-1400 mm (about 47-55 inches), or about 1323 mm (about 52 inches). The second portion 154 may include slots 156. The second portion 154 may have a length of about 100-500 mm (about 3.9-19.7 inches), or about 200-400 mm (about 7.9-15.7 inches) or about 300 mm (about 11.8 inches). The second portion 154 may have a plurality of discrete zones. For example, the second portion 154 may include a distal end zone 160, a first zone 162, a second zone 164, a third zone 166, and a fourth zone 168. This is not intended to be limiting. More or fewer zones may be included.

The distal end zone 160 may be positioned at or adjacent to the distal end of the second portion 154 of the hypotube region 150 and/or distal of the first zone 162. The distal end zone 160 may have a length of about 0.1-0.8 mm (about 0.004-0.03 inches), or about 0.2-0.5 mm (about 0.008-0.02 inches), or about 0.25-0.40 mm (about 0.01-0.016 inches), or about 0.30 mm (about 0.012 inches). In this example, the distal end zone 160 may be free of slots. This may improve/enhance the ability of the hypotube region 150 to be bonded to other structures of a medical device such as the imaging window region (e.g., the imaging window region 48). In addition or in the alternative, this may improve the robustness of the hypotube region 150, which may aid in handling during assembly of the medical device. In some instances, the distal end zone 160 may represent the part of the second portion 154 of the hypotube region 150 that is uncut prior to slots 156 formed in the second portion 154 of the hypotube region 150.

The first zone 162 may be disposed proximal of the distal end zone 160. In at least some instances, the slots 156 in the first zone 162 may have a constant pitch (e.g., where the pitch is understood to be similar to the pitch 76 as defined in FIG. 4). For example, the slots 156 along the first zone 162 may have a pitch of about 0.1-0.35 mm (about 0.004-0.014 inches), or about 0.15-0.25 mm (about 0.006-0.01 inches), or about 0.2-0.25 mm (about 0.008-0.01 inches), or about 0.223 mm (about 0.009 inches).

The cut angle (e.g., where the cut angle is understood to be similar to the cut angle 72 as defined in FIG. 4) of the slots 156 may be about 140-170 degrees, or about 150-160 degrees, or about 155 degrees. In at least some instances, the cut angle may be constant along the length of the first zone 162. The uncut angle (e.g., where the uncut angle is understood to be similar to the uncut angle 74 as defined in FIG. 4) of the slots 156 may be about 40-60 degrees, or about 45-55 degrees, or about 50 degrees. In at least some instances, the uncut angle may be constant along the length of the first zone 162.

The first zone 162 may have a length of about 20-60 mm (about 0.787-2.236 inches), or about 30-50 mm (about 1.181-1.969 inches), or about 40 mm (about 1.575 inches). The kerf width (e.g., where the kerf width is understood to be similar to the kerf width 78 as defined in FIG. 4) may be about 0.005-0.04 mm (about 0.0002-0.002 inches), or about 0.01-0.03 mm (about 0.0004-0.001 inches), or about 0.02 mm (e.g., 0.02+/−0.01 mm).

The second zone 164 may be disposed proximal of the first zone 162. In at least some instances, the slots 156 in the second zone 164 may have a pitch (e.g., where the pitch is understood to be similar to the pitch 76 as defined in FIG. 4) that varies along the length of the second zone 164. For example, the slots 156 adjacent to the distal end of the second zone 164 may have a pitch of about 0.1-0.35 mm (about 0.004-0.014 inches), or about 0.15-0.25 mm (about 0.006-0.01 inches), or about 0.2-0.25 mm (about 0.008-0.01 inches), or about 0.223 mm (about 0.009 inches). The slots 156 adjacent to the proximal end of the second zone 164 may have a pitch of about 0.1-0.4 mm (about 0.0004-0.016 inches), or about 0.15-0.35 mm (about 0.006-0.014 inches), or about 0.2-0.3 mm (about 0.008-0.012 inches), or about 0.279 mm (about 0.011 inches). The change/progression in pitch may be a linear change, a non-linear change, etc. In one example, the progression of the pitch may be a function defined as Pᵢ₊₁=Pᵢ+0.00020 mm (7.87E-6 inches), where Pᵢ is understood to be the initial pitch or pitch at an initial cut position and Pᵢ₊₁ is understood to be the pitch at an immediately adjacent cut.

The cut angle (e.g., where the cut angle is understood to be similar to the cut angle 72 as defined in FIG. 4) of the slots 156 may be about 140-170 degrees, or about 150-160 degrees, or about 155 degrees. In at least some instances, the cut angle may be constant along the length of the second zone 164. The uncut angle (e.g., where the uncut angle is understood to be similar to the uncut angle 74 as defined in FIG. 4) of the slots 156 may be about 40-60 degrees, or about 45-55 degrees, or about 50 degrees. In at least some instances, the uncut angle may be constant along the length of the second zone 164.

The second zone 164 may have a length of about 20-60 mm (about 0.787-2.236 inches), or about 30-50 mm (about 1.181-1.969 inches), or about 40 mm (about 1.575 inches). The kerf width (e.g., where the kerf width is understood to be similar to the kerf width 78 as defined in FIG. 4) may be about 0.005-0.04 mm (about 0.0002-0.002 inches), or about 0.01-0.03 mm (about 0.0004-0.001 inches), or about 0.02 mm (e.g., 0.02+/−0.01 mm).

The third zone 166 may be disposed proximal of the second zone 164. In at least some instances, the slots 156 in the third zone 166 may have a pitch (e.g., where the pitch is understood to be similar to the pitch 76 as defined in FIG. 4) that is constant along the length of the third zone 166. For example, the slots 156 along the third zone 166 may have a pitch of about 0.2-0.6 mm (about 0.008-0.024 inches), or about 0.3-0.5 mm (about 0.012-0.020 inches), or about 0.4 mm (about 0.016 inches).

The cut angle (e.g., where the cut angle is understood to be similar to the cut angle 72 as defined in FIG. 4) of the slots 156 may vary along the length of the third zone 166. For example, the slots 156 adjacent to the distal end of the third zone 166 may have a cut angle of about 130-160 degrees, or about 140-150 degrees, or about 145 degrees. The slots 156 adjacent to the proximal end of the third zone 166 may have a cut angle of about 60-80 degrees, or about 70-75 degrees, or about 72.55 degrees. The change/progression in the cut angle may be a linear change, a non-linear change, etc. In one example, the progression of the cut angle may be a function defined as Cᵢ₊₁=Cᵢ−0.05785 degrees, where Cᵢ is understood to be the initial cut angle or the cut angle at an initial cut position and Cᵢ₊₁ is understood to the cut angle at an immediately adjacent cut.

The uncut angle (e.g., where the uncut angle is understood to be similar to the uncut angle 74 as defined in FIG. 4) of the slots 156 may vary along the length of the third zone 166. For example, the slots 156 adjacent to the distal end of the third zone 166 may have an uncut angle of about 10-30 degrees, or about 15-25 degrees, or about 19 degrees. The slots 156 adjacent to the proximal end of the third zone 166 may have an uncut angle of about 50-80 degrees, or about 60-70 degrees, or about 65 degrees. The change/progression in the uncut angle may be a linear change, a non-linear change, etc. In one example, the progression of the cut angle may be a function defined as Uᵢ₊₁=Uᵢ+0.03680 degrees, where Uᵢ is understood to be the initial uncut angle or the uncut angle at an initial cut position and Uᵢ₊₁ is understood to the uncut angle at an immediately adjacent cut.

The third zone 166 may have a length of about 180-240 mm (about 7.087-9.449 inches), or about 200-220 mm (about 7.874-8.661 inches), or about 210 mm (about 8.268 inches). The kerf width (e.g., where the kerf width is understood to be similar to the kerf width 78 as defined in FIG. 4) may be about 0.005-0.04 mm (about 0.0002-0.002 inches), or about 0.01-0.03 mm (about 0.0004-0.001 inches), or about 0.02 mm (e.g., 0.02+/−0.01 mm).

The fourth zone 168 may be disposed proximal of the third zone 166. In at least some instances, the slots 156 in the fourth zone 168 may have a pitch (e.g., where the pitch is understood to be similar to the pitch 76 as defined in FIG. 4) that varies along the length of the fourth zone 168. For example, the slots 156 adjacent to the distal end of the fourth zone 168 may have a pitch of about 0.2-0.6 mm (about 0.008-0.024 inches), or about 0.3-0.5 mm (about 0.012-0.020 inches), or about 0.4 mm (about 0.016 inches). The slots 156 adjacent to the proximal end of the fourth zone 168 may have a pitch of about 0.4-0.8 mm (about 0.016-0.031 inches), or about 0.5-0.7 mm (about 0.020-0.028 inches), or about 0.6 mm (about 0.024 inches). The change/progression in pitch may be a linear change, a non-linear change, etc. In one example, the progression of the pitch may be a function defined as Pᵢ₊₁=Pᵢ+0.00385 mm (1.51E-4 inches), where Pᵢ is understood to be the initial pitch or pitch at an initial cut position and Pᵢ₊₁ is understood to be the pitch at an immediately adjacent cut.

The cut angle (e.g., where the cut angle is understood to be similar to the cut angle 72 as defined in FIG. 4) of the slots 156 may vary along the length of the fourth zone 168. For example, the slots 156 adjacent to the distal end of the fourth zone 168 may have a cut angle of about 60-80 degrees, or about 70-75 degrees, or about 72.55 degrees. The slots 156 adjacent to the proximal end of the fourth zone 168 may have a cut angle of about 30-60 degrees, or about 40-50 degrees, or about 44 degrees. The change/progression in the cut angle may be a linear change, a non-linear change, etc. In one example, the progression of the cut angle may be a function defined as Cᵢ₊₁=Cᵢ−0.54904 degrees, where Cᵢ is understood to be the initial cut angle or the cut angle at an initial cut position and Cᵢ₊₁ is understood to the cut angle at an immediately adjacent cut.

The uncut angle (e.g., where the uncut angle is understood to be similar to the uncut angle 74 as defined in FIG. 4) of the slots 156 may vary along the length of the fourth zone 168. For example, the slots 156 adjacent to the distal end of the fourth zone 168 may have an uncut angle of about 50-80 degrees, or about 60-70 degrees, or about 65 degrees. The slots 156 adjacent to the proximal end of the fourth zone 168 may have an uncut angle of about 70-100 degrees, or about 80-95 degrees, or about 88 degrees. The change/progression in the uncut angle may be a linear change, a non-linear change, etc. In one example, the progression of the cut angle may be a function defined as Uᵢ₊₁=Uᵢ+0.44231 degrees, where Uᵢ is understood to be the initial uncut angle or the uncut angle at an initial cut position and Uᵢ₊₁ is understood to the uncut angle at an immediately adjacent cut.

The fourth zone 168 may have a length of about 1-20 mm, or about 5-15 mm, or about 10 mm. The kerf width (e.g., where the kerf width is understood to be similar to the kerf width 78 as defined in FIG. 4) may be about 0.005-0.04 mm (about 0.0002-0.002 inches), or about 0.01-0.03 mm (about 0.0004-0.001 inches), or about 0.02 mm (e.g., 0.02+/−0.01 mm).

The materials that can be used for the various components of the medical device 10 (and/or other devices disclosed herein) and the various tubular members disclosed herein may include those commonly associated with medical devices. For simplicity purposes, the following discussion refers to the elongate shaft 12 and other components of the medical device 10. However, this is not intended to limit the devices and methods described herein, as the discussion may be applied to other similar tubular members and/or components of tubular members or devices disclosed herein.

The elongate shaft 12 and/or other components of the medical device 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the medical device 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device 10 to achieve the same result.

In some embodiments, a degree of Magnetic Resonance Imaging (MRI) compatibility is imparted into the medical device 10. For example, the medical device 10, or portions thereof, may be made of a material that does not substantially distort the image and create substantial artifacts (e.g., gaps in the image). Certain ferromagnetic materials, for example, may not be suitable because they may create artifacts in an MRI image. The medical device 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An intravascular imaging device, comprising:
a catheter shaft including a hypotube region and an imaging window region;
wherein the hypotube region includes a slotted section having a plurality of slots formed therein;
wherein the slotted section includes a plurality of discrete zones including a first zone, a second zone disposed proximal of the first zone, a third zone disposed proximal of the second zone, a fourth zone disposed proximal of the third zone;
wherein the slots along the first zone have a constant first zone pitch;
wherein the slots along the second zone have a second zone pitch that varies along a second zone length;
wherein the slots along the third zone have a constant third zone pitch;
wherein the slots along the fourth zone have a fourth zone pitch that varies along a fourth zone length; and
an imaging core disposed within the catheter shaft.

2. The intravascular imaging device of claim 1, further comprising a distal end zone disposed distally of the first zone.

3. The intravascular imaging device of claim 2, wherein the distal end zone is free of slots.

4. The intravascular imaging device of claim 1, wherein the slots along the first zone have a constant first cut angle.

5. The intravascular imaging device of claim 4, wherein the constant first cut angle is about 150-160 degrees.

6. The intravascular imaging device of claim 1, wherein the slots along the second zone have a constant second cut angle.

7. The intravascular imaging device of claim 6, wherein the constant second cut angle is about 150-160 degrees.

8. The intravascular imaging device of claim 1, wherein the slots along the third zone have a cut angle that varies along a third zone length thereof.

9. The intravascular imaging device of claim 8, wherein the cut angle of the slots adjacent to a distal end of the third zone is about 140-150 degrees and wherein the cut angle of the slots adjacent to a proximal end of the third zone is about 70-75 degrees.

10. The intravascular imaging device of claim 1, wherein the slots of the fourth zone have a cut angle that varies along a fourth zone length thereof.

11. The intravascular imaging device of claim 10, wherein the cut angle of the slots adjacent to a distal end of the fourth zone is about 70-75 degrees and wherein the cut angle of the slots adjacent to a proximal end of the fourth zone is about 40-50 degrees.

12. An intravascular imaging device, comprising:
a catheter shaft including a hypotube region, an imaging window region, and a distal end region having a guidewire lumen formed therein;
wherein the hypotube region includes a slotted section having a plurality of slots formed therein;
wherein the slotted section includes a plurality of discrete zones including a first zone, a second zone disposed proximal of the first zone, a third zone disposed proximal of the second zone, a fourth zone disposed proximal of the third zone;
wherein the slots along the first zone have a constant first cut angle;
wherein the slots along the second zone have a constant second cut angle;
wherein the slots along the third zone have a third cut angle that varies along a third zone length thereof;
wherein the slots along the fourth zone have a fourth cut angle that varies along a fourth zone length thereof; and
an imaging core disposed within the catheter shaft.

13. The intravascular imaging device of claim 12, wherein the slots along the first zone have a constant first zone pitch.

14. The intravascular imaging device of claim 12, wherein the slots along the second zone have a second zone pitch that varies along a second zone length.

15. The intravascular imaging device of claim 12, wherein the slots along the third zone have a constant third zone pitch.

16. The intravascular imaging device of claim 12, wherein the slots along the fourth zone have a fourth zone pitch that varies along a fourth zone length.

17. The intravascular imaging device of claim 12, wherein the third cut angle of the slots adjacent to a distal end of the third zone is about 140-150 degrees and wherein the third cut angle of the slots adjacent to a proximal end of the third zone is about 70-75 degrees.

18. The intravascular imaging device of claim 12, wherein the fourth cut angle of the slots adjacent to a distal end of the fourth zone is about 70-75 degrees and wherein the fourth cut angle of the slots adjacent to a proximal end of the fourth zone is about 40-50 degrees.

19. The intravascular imaging device of claim 12, further comprising a distal end zone disposed distally of the first zone, the distal end zone being free of slots.

20. A method for imaging a blood vessel, the method comprising:
disposing an intravascular imaging device within a blood vessel, the intravascular imaging device comprising:
a catheter shaft including a hypotube region and an imaging window region,
wherein the hypotube region includes a slotted section having a plurality of slots formed therein,
wherein the slotted section includes a plurality of discrete zones including a first zone, a second zone disposed proximal of the first zone, a third zone disposed proximal of the second zone, a fourth zone disposed proximal of the third zone,
wherein the slots along the first zone have a constant first zone pitch,
wherein the slots along the second zone have a second zone pitch that varies along a second zone length,
wherein the slots along the third zone have a constant third zone pitch,
wherein the slots along the fourth zone have a fourth zone pitch that varies along a fourth zone length, and
an imaging core disposed within the catheter shaft; and
translating the imaging core relative to the catheter shaft.
